(19) United States
(12) Patent Application Publication
(10) Pub. No.: US 2019/0162735 A1
(43) Pub. Date: May 30, 2019

(54) BIOMOLECULE EXTRACTION METHOD, EXTRACTION SYSTEM, AND EXTRACTION CONTAINER

(71) Applicant: Panasonic Intellectual Property Management Co., Ltd., Osaka (JP)

(72) Inventors: CHIAKI SHIDO, Kyoto (JP); HIROYUKI TANAKA, Nara (JP)

(21) Appl. No.: 16/323,530

(22) PCT Filed: Sep. 25, 2017
(86) PCT No.: PCT/JP2017/034420

§ 371 Date: Feb. 6, 2019

(30) Foreign Application Priority Data
Oct. 13, 2016 (JP) 2016-201719

(51) Int. Cl. G01N 33/68

(52) U.S. Cl. CPC G01N 33/6881; G01N 2800/20

(57) ABSTRACT

An object of the present disclosure is to provide a method and a system capable of extracting biomolecules simply and highly reproducibly by using skin tissue collected with a tape stripping method. The biomolecule extraction method according to the present disclosure is a method for extracting biomolecules contained in skin tissue by using an extraction reagent and a magnetic substance in an extraction container, and has the following features. That is, a tape that has collected the skin tissue is brought into contact with the extraction reagent and the magnetic substance, the magnetic substance in the extraction container is moved by magnetic force generated from a magnet provided outside the extraction container, and the biomolecules are extracted from the skin tissue into the extraction reagent by mechanical friction between the magnetic substance and the tape adhered with the skin tissue.

### TECHNICAL FIELD

The present disclosure relates to a method and a system for extracting biomolecules contained in skin tissue collected with a tape.

### BACKGROUND ART

In a field of dermatology, examples of a method for evaluating a state of skin include protein analysis. As the method for analyzing protein in skin, a colorimetric method and an antigen-antibody method have been known, for example (NPLs 1, 2). To analyze protein, it needs to be collected from skin tissue. Examples of the method for collecting protein from skin tissue include a tape stripping method (PTL 1). The tape stripping method is a noninvasive method for collecting protein by peeling off a part of skin tissue by using an adhesive tape stuck on skin. Protein is then extracted from the collected skin tissue. With the tape stripping method, collection of the skin tissue can be performed relatively simply. Further, this method is excellent in that a load imposed on a subject is small. Therefore, in recent years, collection of the skin tissue using the tape stripping method has attracted attention in, for example, a skin inspection for general consumers in which imposing a load on the skin of a subject is not acceptable.

### CITATION LIST

### Patent Literature

PTL 1: Unexamined Japanese Patent Publication No. 2008-249429

### Non-Patent Literature

NPL 1: I. L. A. Boxman, P. J. Hensbergen, R. C. Van Der Schors, D. P. Bruynzeel, C. P. Tensen and M. Ponec, Br. J. Dermatol., 146, 777-785 (2002)

NPL 2: Stephen M. Keyse and Rex M. Tyrrell, Proc. Natl. Acad. Sci., 86, 99-103 (1989)

### SUMMARY OF THE INVENTION

However, the method for extracting protein from the skin tissue collected with the tape stripping method has problems in which manipulation of a user who performs the extraction is complicated, and therefore an extracted amount of protein varies widely. An object of the present disclosure is to provide a method and a system capable of extracting protein from skin tissue collected by the tape stripping method simply and highly reproducibly.

A biomolecule extraction method according to the present disclosure is a method for extracting biomolecules contained in skin tissue by using an extraction reagent and a magnetic substance in an extraction container, and has the following features. That is, a tape that has collected the skin tissue is brought into contact with the extraction reagent and the magnetic substance, the magnetic substance in the extraction container is moved by magnetic force generated by a magnet provided outside the extraction container, and the biomolecules are extracted from the skin tissue into the extraction reagent by mechanical friction between the magnetic substance and the tape adhered with the skin tissue.

The biomolecule extraction method according to the present disclosure can simply and highly reproducibly extract biomolecules from the skin tissue collected using the tape.

### BRIEF DESCRIPTION OF DRAWINGS

FIG. 1 is a schematic perspective view illustrating an extraction device of protein according to the present exemplary embodiment.

FIG. 2 is a schematic sectional view illustrating an extraction system of protein according to the present exemplary embodiment.

FIG. 3 is a schematic top view illustrating the extraction system of protein according to the present exemplary embodiment.

FIG. 4 is a schematic top perspective view illustrating an extraction container of protein according to the present exemplary embodiment.

FIG. 5 is a flow chart illustrating an extraction method of protein according to the present exemplary embodiment.

FIG. 6 is a view illustrating a scanning electron microscope (SEM) image of polylactic acid beads in Example 1.

FIG. 7 is a graph illustrating an extracted amount of protein in Example 1.

FIG. 8 is a view illustrating a SEM image of magnetic ferritic stainless steel in Example 2.

FIG. 9 is a graph illustrating an extracted amount of protein in Example 2.

FIG. 10 is a graph illustrating an extracted amount of protein in Example 3.

FIG. 11 is a sectional view illustrating another example of the extraction system of protein according to the present exemplary embodiment.

FIG. 12 is a sectional view illustrating still another example of the extraction system of protein according to the present exemplary embodiment.

### DESCRIPTION OF EMBODIMENT

Hereinafter, a biomolecule extraction method and a biomolecule extraction system according to an exemplary embodiment of the present disclosure will be described in detail with reference to the drawings. Note that the exemplary embodiment described below illustrates a preferred specific example of the present disclosure. Accordingly, numerical values, shapes, materials, constituent elements, disposition and connection modes of the constituent elements, and the like illustrated in the following exemplary embodiment are merely examples, and therefore are not intended to limit the present disclosure. Therefore, among constituent elements in the following exemplary embodiment, constituent elements not recited in the independent claim indicating the broadest concept of the present invention are described as optional constituent elements.

Also, the drawings are schematic views, and are not necessarily strictly accurate. In the drawings, substantially identical structural elements are denoted by like reference signs and repetitive explanations thereof will be omitted or simplified.

### Exemplary Embodiment

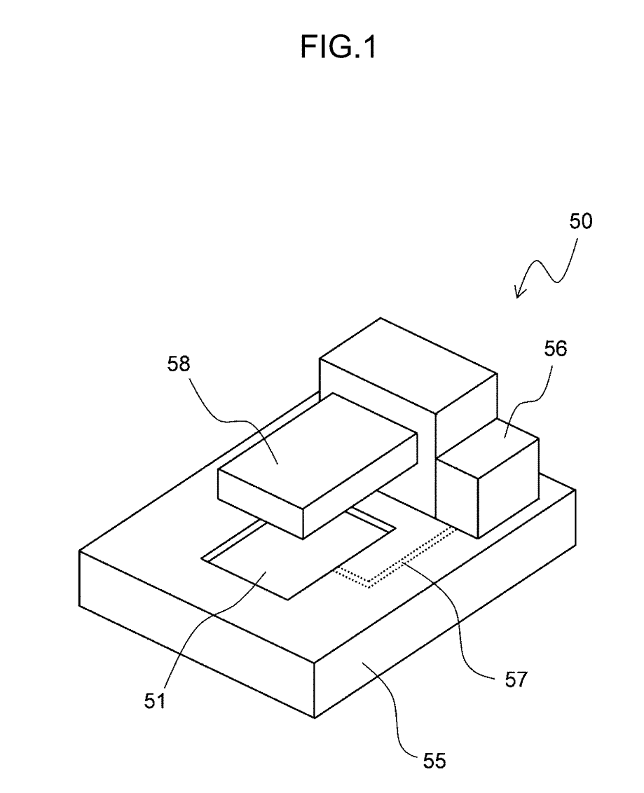

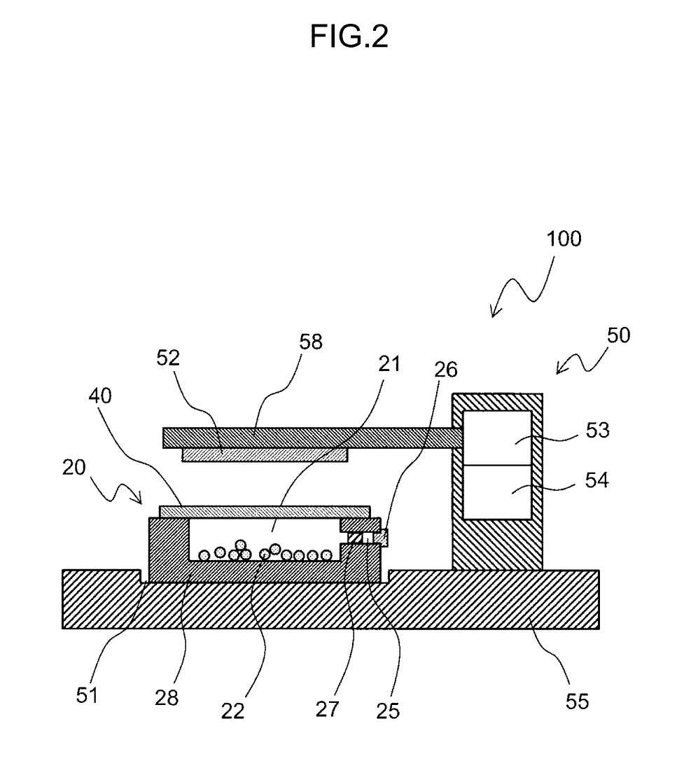

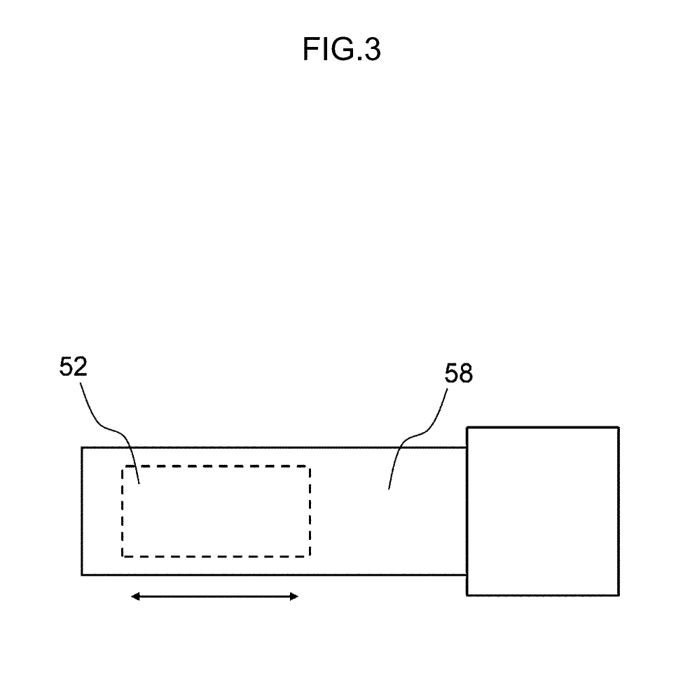

FIG. 1 is a schematic perspective view illustrating a biomolecule extraction system according to the present exemplary embodiment. FIG. 2 is a schematic sectional view illustrating the biomolecule extraction system according to the present exemplary embodiment. FIG. 3 is a schematic top view illustrating the biomolecule extraction system according to the present exemplary embodiment.

Biomolecule extraction system 100 includes extraction container 20 and extraction device 50.

Note that the biomolecule is protein, a lipid, or deoxyribo nucleic acid (DNA) of normal bacteria flora, which is extracted from a horny layer, for example. Hereinafter, the description will be made assuming that the biomolecule is protein contained in the horny layer.

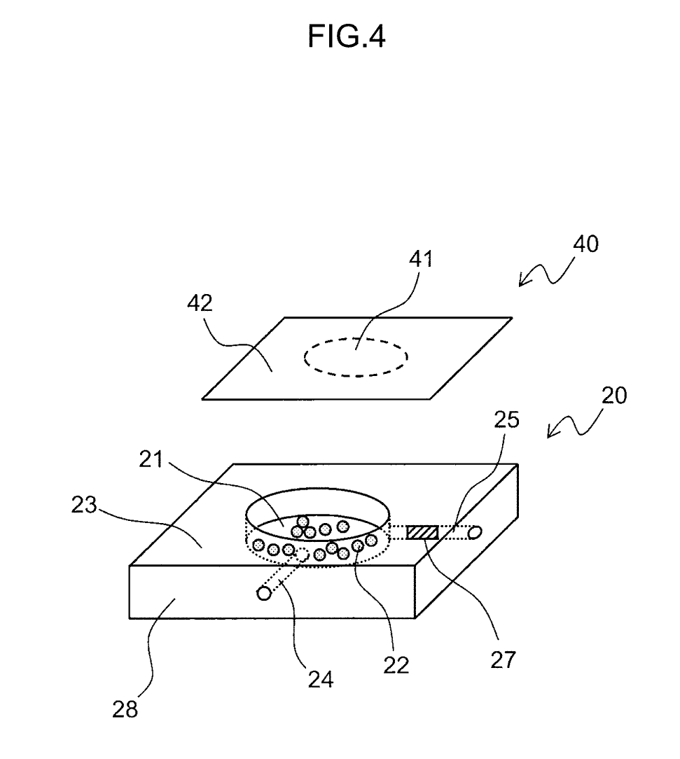

FIG. 4 is a schematic perspective view illustrating extraction container 20 in the present exemplary embodiment.

Extraction container 20 includes extraction unit 21, magnetic bodies 22 disposed in extraction unit 21, and sticking surface 23 to be stuck with collection tape 40 that has collected skin tissue. Extraction container 20 further includes injection path 24 and recovery path 25 connected to extraction unit 21. An extraction reagent is injected into extraction unit 21 through injection path 24. An extraction solution that has extracted protein is recovered through recovery path 25.

Extraction container 20 can be formed by using substrate 28 made of a resin material, glass, or a metallic material, for example. For example, the resin material is polymethyl methacrylate (abbreviated as PMMA) or polydimethyl siroxane (abbreviated as PDMS). As the metallic material, stainless steel can be used, for example. Note that, as the metallic material, metal having good biocompatibility, such as titanium or stainless steel for medical use may be used. The material of the extraction container may be another material that does not show a notable reaction to the extraction reagent.

Extraction unit 21 is a recess provided in substrate 28 constituting extraction container 20, for example. Extraction unit 21 has a circular shape in top view. Extraction unit 21 is provided on sticking surface 23 of extraction container 20. In other words, extraction unit 21 is formed at a position to be covered by collection tape 40 to be stuck on sticking surface 23. An extraction reagent of horny layer protein is introduced into the recess. Further magnetic bodies 22 are disposed in the recess. Note that the shape of extraction unit 21 is not limited to be circular. For example, the shape of extraction unit 21 may be a quadrangle or a drop shape in top view.

Magnetic bodies 22 are magnetic beads, for example. A diameter of each magnetic substance 22 is from 50 μm to 2 mm inclusive, for example. The diameter of each magnetic substance 22 is appropriately determined according to a depth of extraction unit 21.

Magnetic bodies 22 may be made of a material that shows magnetism to an approaching magnet, and a ferromagnetic material or a paramagnetic material can be used for magnetic bodies 22. In order not to affect biochemical analysis to be performed later, as magnetic bodies 22, magnetic beads for bio experiments or magnetic stainless steel particles for medical use is preferably used, for example. Those materials have corrosion resistance so that those materials do not produce metal ions in a solution with physiological pH. For magnetic bodies 22, a material that hardly causes nonspecific adsorption with respect to the horny layer protein to be extracted is preferably used. Note that magnetic bodies 22 may be magnetic beads each covered with a polymeric material such as a polymer.

Magnetic bodies 22 are supported at a bottom surface or a side surface of extraction unit 21. Magnetic bodies 22 are preferably fixed to extraction unit 21. Examples of a method for supporting magnetic bodies 22 at a supporting part on the bottom surface or the side surface of extraction unit 21 include a method for supporting using magnetic force and a method for supporting using a water-soluble substance as an adhesive. Herein examples of the water-soluble substance include starch, chitosan, cellulose, and salt. Magnetic bodies 22 fixed to the supporting part are peeled off from the supporting part in an extraction process and drift in the extraction reagent.

Sticking surface 23 is a surface in which an opening is formed in extraction container 20. Collection tape 40 for collecting skin tissue is stuck to sticking surface 23.

Collection tape 40 includes horny layer collection part 41 for collecting skin tissue, and adhesive part 42 for sticking collection tape 40 to sticking surface 23 of extraction container 20. At least one surface of collection tape 40 has adhesiveness, and collection tape 40 is used to collect a horny layer from a surface of skin Collection tape 40 is stuck so as to cover the entire opening of extraction unit 21. Horny layer collection part 41 is provided at a position facing extraction unit 21.

Collection tape 40 is an example of a carrier that can cause skin tissue to adhere to a surface of the carrier. As another form of the carrier, a sheet or a plate that has no adhesive surface on its surface may be used. In this case, since skin tissue is separately collected and is adhered to the carrier, an adhesive layer may be formed on a surface of the carrier. After the skin tissue is adhered to the carrier, the carrier may be stuck on extraction container 20, similar to collection tape 40 described above. Note that it is preferable that the carrier has a flat shape.

Injection path 24 and recovery path 25 are formed in extraction container 20. Injection path 24 and recovery path 25 are connected to extraction unit 21.

Stopper 26 is provided at a discharge port of recovery path 25. Filter 27 is provided in recovery path 25.

Stopper 26 is provided to prevent an extraction solution from being leaked from extraction unit 21.

Filter 27 can separate magnetic bodies 22 from the extraction solution that has extracted horny layer protein. Filter 27 can be made from a nonwoven fabric or a porous body, for example. A size of each void of filter 27 is smaller than a diameter of each magnetic substance 22. By providing filter 27 in recovery path 25, magnetic bodies 22 can easily be separated from the extraction solution. Therefore, protein analysis can easily be performed later.

Filter 27 may be provided to remove residues of the adhesive produced upon extracting protein or insoluble fractions in a horny layer sample. In this case, as a material of filter 27, a material with less adsorption of target protein is appropriately selected and used from among stainless steel, polypropylene, Teflon (registered trademark), and silicon, for example. A hole diameter is also selected in accordance with separation of magnetic bodies 22. Filter 27 can integrally be made of the same material as a material of extraction container 20 upon forming extraction container 20.

Note that magnetic bodies 22 may be attracted to one side of extraction unit 21 by using magnet 52 in extraction device 50, to remove magnetic bodies 22 from the extraction solution without using filter 27.

Extraction device 50 includes placement part 51 for placing extraction container 20, magnet 52 provided at a position facing sticking surface 23 of extraction container 20 placed on placement part 51, drive unit 53 for driving magnet 52, and controller 54 for controlling operations of magnet 52 and drive unit 53. Drive unit 53 includes support 58. Magnet 52 is provided on a lower surface of support 58.

Magnet 52 moves magnetic bodies 22 disposed in extraction container 20. In other words, magnetic bodies 22 are controlled so as to move inside extraction unit 21 through movement of magnet 52. Extraction device 50 extracts protein contained in the horny layer from the skin tissue adhered to collection tape 40 by mechanical friction generated, according to movement of magnet 52, between collection tape 40 to which the skin tissue adheres and magnetic bodies 22 brought into contact with collection tape 40.

Extraction container 20 on which collection tape 40 is stuck is placed on placement part 51. Placement part 51 may be a depression on which extraction container 20 is placed on plate 55, for example.

Magnet 52 is used to control movement of magnetic bodies 22 disposed in extraction unit 21. Magnet 52 may be any magnet having magnetic force. As magnet 52, a neodymium magnet having strong magnetic force is preferably used, for example. Alternatively, magnet 52 may be an electromagnet. The electromagnet can move magnetic bodies 22 through a change of a magnetic field caused based on electrical control. In this case, the electromagnet may not move.

Drive unit 53 moves magnet 52. Magnet 52 is provided on support 58, for example. Support 58 is connected to drive unit 53. Drive unit 53 is, for example, a motor or an actuator.

Controller 54 controls an operation of drive unit 53. In other words, controller 54 controls movement of magnetic bodies 22 through magnet 52. Note that, when the electromagnet is used as magnet 52, controller 54 controls a change of a magnetic field of the electromagnet.

Note that, in extraction system 100, extraction container 20 may be fixed to placement part 51 of extraction device 50. In this case, extraction container 20 constitutes a part of extraction device 50. When extraction container 20 constitutes a part of extraction device 50, extraction container 20 may not be disposable, but may be repeatedly used while being washed.

Reagent tank 56 can accommodate the extraction reagent to be injected into extraction unit 21. Reagent tank 56 is connected to extraction unit 21 through passage 57 and injection path 24. The extraction reagent is injected into extraction unit 21 through passage 57 and injection path 24. A material of reagent tank 56 may be any material having corrosion resistance against the extraction reagent.

Note that, when magnetic bodies 22 are put into extraction unit 21 in a process of extracting protein, magnetic bodies 22 may not be supported with extraction unit 21 in advance. Further, when a user manually injects and recovers the extraction reagent and the extraction solution in the process of extracting protein, extraction container 20 may not include injection path 24 and recovery path 25.

Hereinafter, a method for extracting protein contained in a horny layer will be described.

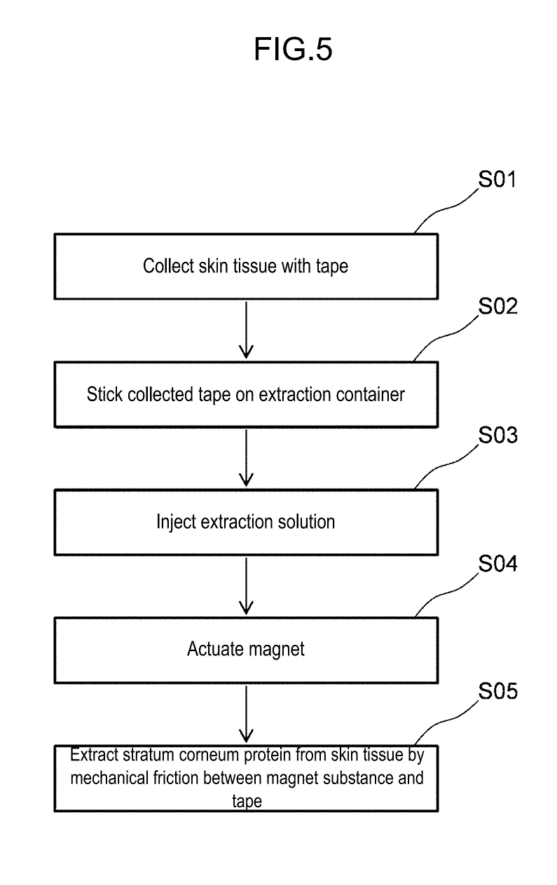

FIG. 5 is a flowchart illustrating the method for extracting protein.

The user peels off a protection tape covering horny layer collection part 41, and sticks on skin. At this time, it is desirable that make-up on the skin is removed, and the skin is cleaned by using, for example, 70% ethanol in advance. The user sticks collection tape 40 on a portion to be analyzed of the skin. Subsequently, the user rubs entire horny layer collection part 41 of collection tape 40 with uniform force for about 30 seconds, and then peels off collection tape 40 gently. Skin tissue containing horny layer protein adheres to horny layer collection part 41 of collection tape 40. This allows the user to noninvasively collect the horny layer on a surface of the skin (S01).

Next, collection tape 40 that has collected the skin tissue is stuck on sticking surface 23 of extraction container 20 (S02). Collection tape 40 seals the opening of extraction unit 21 of extraction container 20. Horny layer collection part 41 adhered with the skin tissue is positioned at the opening formed on sticking surface 23, and faces extraction unit 21.

The extraction reagent is injected into extraction unit 21 with collection tape 40 being stuck on extraction container 20 (S03). At this time, the extraction reagent is injected into extraction unit 21 until the extraction reagent comes into contact with horny layer collection part 41 of collection tape 40 and the skin tissue. It is desirable that extraction unit 21 is completely filled with the extraction solution. At this time, magnetic bodies 22 fixed to extraction unit 21 are released from the supporting part.

Magnet 52 of extraction device 50 is then moved closer to collection tape 40 of extraction container 20 to operate (S04). At this time, magnetic bodies 22 in extraction unit 21 are moved in the extraction reagent, and are attracted to magnet 52. Magnet 52 is moved in parallel with collection tape 40, for example. Note that magnet 52 moves linearly as illustrated with an arrow in figure, for example. Alternatively, magnet 52 may be moved circularly, in a meandering manner, or three-dimensionally.

Magnet 52 is disposed on an opposite side of collection tape 40 from magnetic bodies 22. Accordingly, magnetic bodies 22 attracted to magnet 52 are brought into contact with collection tape 40. By moving magnet 52, magnetic bodies 22 are moved along a surface, of collection tape 40, to which the skin tissue is adhered while being in contact with collection tape 40. At this time, mechanical friction is generated between magnetic bodies 22 and collection tape 40. This mechanical friction force peels off horny layer protein from the skin tissue adhered to collection tape 40 to be dissolved in the extraction reagent. Horny layer protein can thus be extracted.

Note that collection tape 40 may be included in extraction unit 21, for example. For example, a surface opposite to a surface, of collection tape 40, on which horny layer collection part 41 is provided may be stuck on a side surface or a bottom surface of extraction unit 21. In this case, magnet 52 is moved from an outer side of the surface of extraction container 20 on which collection tape 40 is stuck to come closer to the surface, so that magnetic bodies 22 and collection tape 40 in extraction unit 21 can be brought into contact with each other. Accordingly, the mechanical friction is generated between magnetic bodies 22 and collection tape 40, so that horny layer protein can be extracted from the skin tissue adhered to collection tape 40.

The extraction reagent may be injected into extraction container 20 before collection tape 40 is stuck on extraction container 20. At this time, magnetic bodies 22 may be put into extraction unit 21 concurrently with the extraction reagent. Also in this case, it is preferable that extraction unit 21 is completely filled with the extraction reagent.

Hereinafter, the present disclosure will further be described with reference to specific examples. Note that the following examples are examples for describing the present disclosure in more detail, and the exemplary embodiment is not limited to the following examples.

### EXAMPLE 1

An extraction test of protein using magnetic polylactic acid beads as the magnetic bodies is described below.

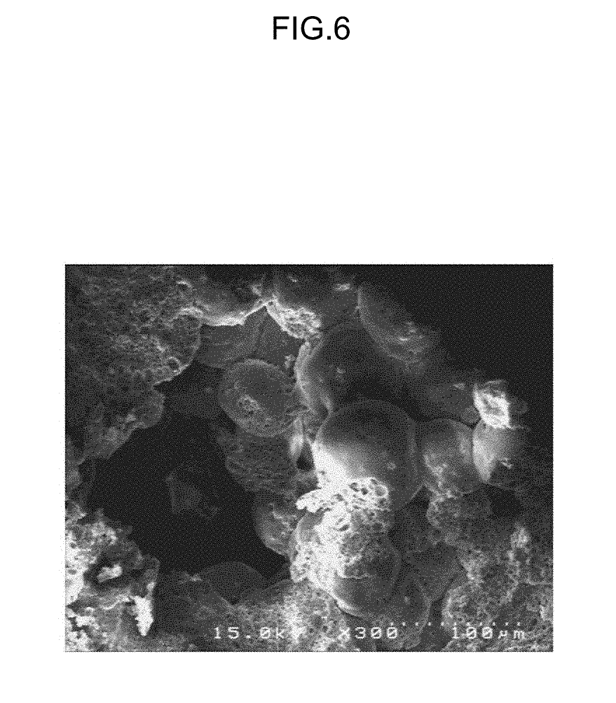

Example 1 is described below. In Example 1, horny layer protein was extracted using extraction container 20 without injection path 24 and recovery path 25. Horny layer checker (product name) manufactured by PROMOTOOL CORPORATION was used as collection tape 40. As extraction container 20, a microtiter plate made of polystyrene with 96 wells is used. Wells (each of which has a diameter of about 7 mm) in the plate served as extraction unit 21. As magnet 52, a neodymium magnet having a rectangular parallelepiped shape with a length of 1 cm, a width of 1 cm, and a height of 2 cm (1 cm×1 cm×2 cm) was used. As magnetic bodies 22, magnetic polylactic acid beads whose average particle diameter is 100 μm were used. FIG. 6 is a SEM image of the magnetic polylactic acid beads used in Example 1. Magnetic bodies 22 were supported in extraction unit 21 by magnetic force, and after extraction unit 21 is filled with the extraction reagent, magnetic bodies 22 were released into the extraction reagent.

As the extraction reagent to be introduced into extraction unit 21, a tris-hydrochloric acid buffer solution (Tris-HCl) (pH: 8.0) added with sodium chloride (NaCl) and sodium dodecyl sulfate (SDS) was used. Note that, in final concentration of the extraction reagent, Tris-HCl was 150 mM, NaCl was 120 mM, and SDS was 1% (w/v). The magnetic polylactic acid beads were supported so as to be 10⁴ pieces/well.

First, collection tape 40 being subjected to masking was stuck on a skin on an inner side of an elbow wiped and cleaned with an ethanol solution of 70% (v/v), and a surface of the tape was rubbed for about 10 seconds. Then collection tape 40 was peeled off from the skin, and was stuck on a plate, which is previously filled with the extraction reagent and magnetic bodies 22, to seal the wells. At this time, adhesive strength may be increased by sticking another adhesive tape on collection tape 40. Note that, in the above description, mM is an abbreviation of mmol/L, and indicates an amount of substance per liter (milli-mol number). Further, w/v indicates (mass of a solute)/(volume of a solution), and v/v indicates (volume of a solute)/(volume of a solution).

Next, the neodymium magnet was linearly moved on the wells sealed by collection tape 40 in parallel with collection tape 40 at a rate of about 100 reciprocations per minute. With this configuration, magnetic bodies 22 in extraction unit 21 were moved so as to be brought into contact with collection tape 40. A time period for moving magnet 52 was set to be one minute, three minutes, and five minutes.

After horny layer protein was extracted, collection tape 40 was peeled off gently from the plate, and an extraction solution of protein was recovered by using a micropipette. An amount of extracted horny layer protein was quantified using a bicinchoninic acid (abbreviated as BCA) method, in terms of bovine serum albumin (abbreviated as BSA).

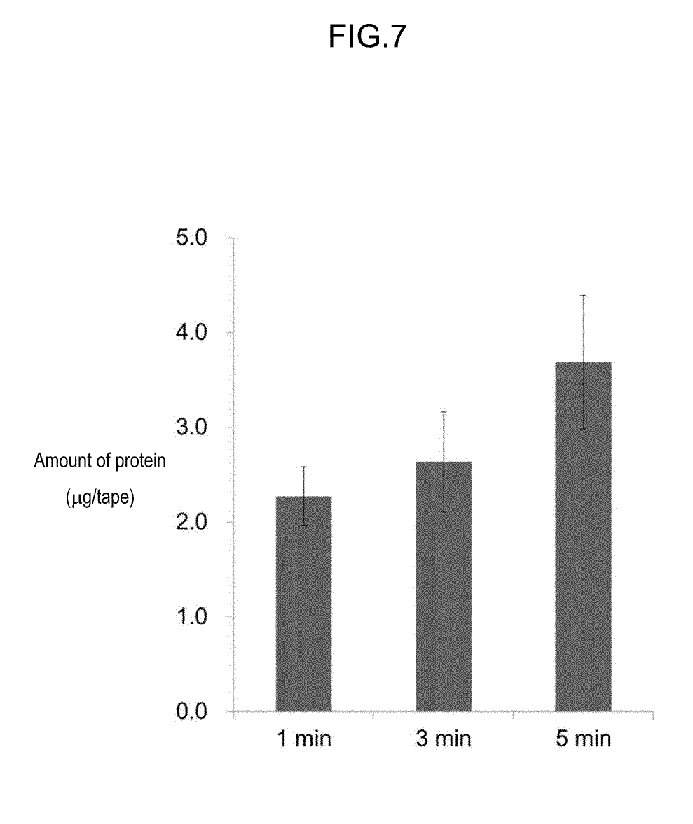

FIG. 7 is a graph indicating experimental results in Example 1. With reference to FIG. 7, horny layer protein of 2 μg to 4 μg was extracted in one collection tape 40. With increasing time for moving magnet 52, an extracted amount of protein was increased.

### EXAMPLE 2

The extraction test of protein using magnetic ferritic stainless steel as the magnetic bodies is described below.

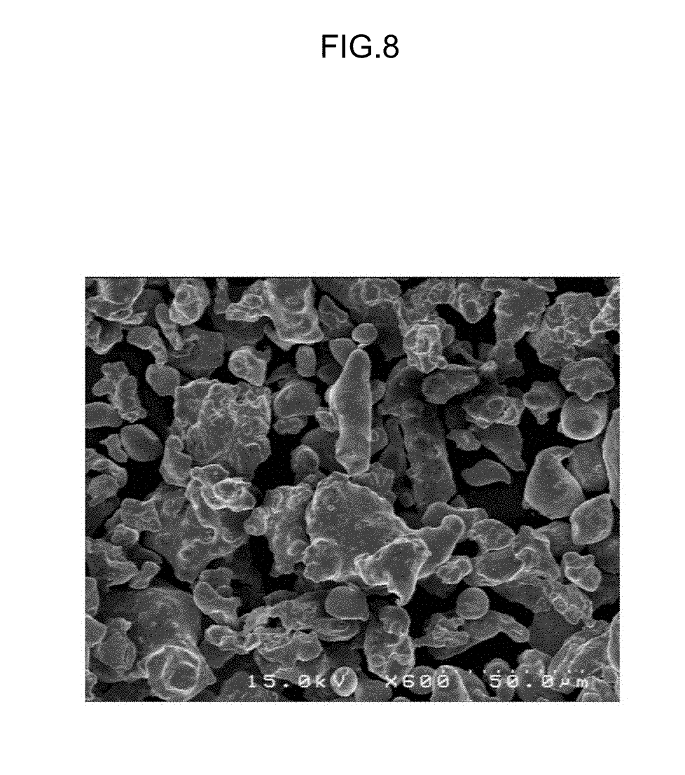

Example 2 was performed according to Example 1. Note that magnetic ferritic stainless steel particles whose average particle diameter is 70 μm were used as magnetic bodies 22. FIG. 8 is a SEM image of the magnetic ferritic stainless steel particles used in Example 2.

Extraction unit 21 was filled with the magnetic ferritic stainless steel particles such that the magnetic ferritic stainless steel particles amount to 10% (w/v). A time period for moving magnet 52 was set to be one minute, three minutes, and five minutes.

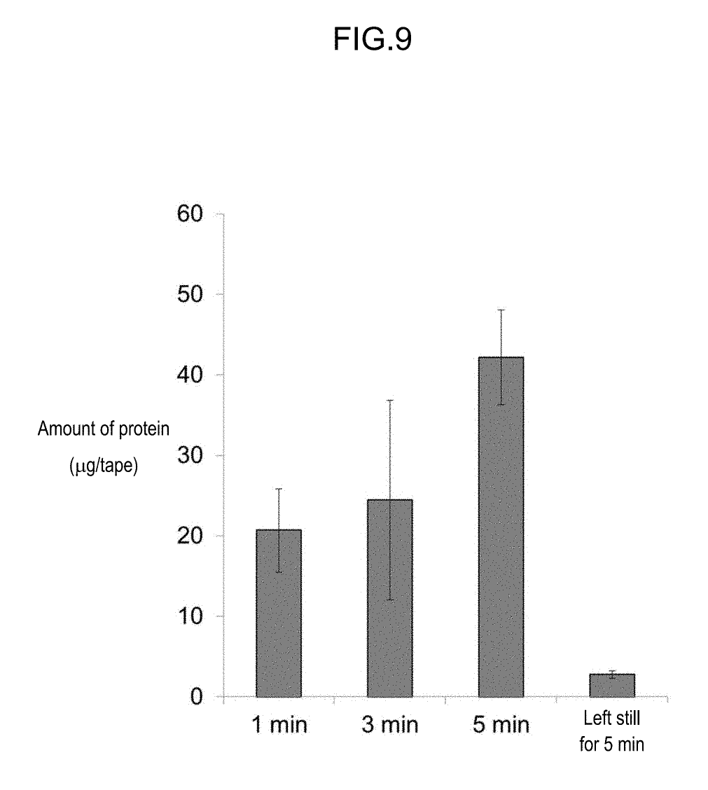

FIG. 9 illustrates experimental results. With reference to FIG. 9, with increasing time for moving magnet 52, an extracted amount of protein was increased. Further, based on comparison between FIG. 7 and FIG. 9, an extracted amount with the magnetic ferritic stainless steel particles was larger than an extracted amount with the magnetic polylactic acid beads. From the SEM images illustrated in FIG. 6 and FIG. 8, the polylactic acid beads each have a circular shape, whereas the magnetic ferritic stainless steel particles each have a shape having protrusions on its surface. Some protrusions on surfaces of the magnetic ferritic stainless steel particles are sharp. Accordingly, it is conceivable that those sharp shapes of the magnetic ferritic stainless steel particles produce an effect for effectively peeling off cells in the horny layer from the tape, and as a result, this effect is one factor for obtaining a larger extracted amount.

### EXAMPLE 3

The extraction test of protein using an extraction chamber made of PMMA is described below.

Example 3 was performed according to Example 2. Note that horny layer checker manufactured by ASAHI BIOMED Co, Ltd. was used as collection tape 40. Further, the extraction chamber made of PMMA was used as extraction container 20. The extraction chamber made of PMMA was formed by cutting a plate of PMMA with a thickness of 2 mm, and cut plates were stuck to each other with an adhesive of methylene dichloride. Extraction unit 21 has a shape with an opening diameter of 2 cm and a height of 2 mm. Note that the shape of the chamber is not limited thereto, but can be modified appropriately according to a volume of extraction liquid or a design of a micro fluid chip to be formed.

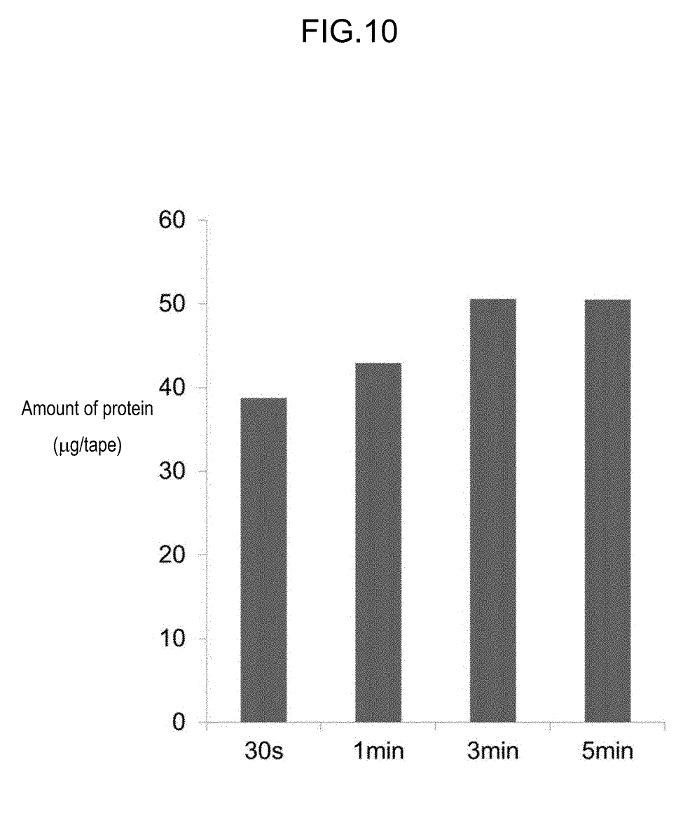

FIG. 10 illustrates experimental results in Example 3. With reference to FIG. 10, with increasing time for moving the magnet, an extracted amount of protein was increased. The extracted amount was substantially equal with three minutes and five minutes. Therefore, it is conceivable that, in this condition, a sufficient amount of protein can be extracted with processing continued for three minutes or more. The extracted amount of protein in Example 3 was larger than that in Example 2 on average. This is because a larger opening in extraction unit 21 can increase an area of the horny layer collection tape, thereby increasing the extracted amount of protein.

Note that, to detect a small amount of protein, the extraction solution is preferably condensed before the detection is performed. Alternatively, by decreasing a volume of extraction unit 21, the small amount of protein can sensitively be detected, without performing the condensation. To decrease the volume of extraction unit 21, a depth of extraction unit 21 may be reduced, for example.

Next, another example of the extraction system of horny layer protein is described.

Configurations similar to those in extraction system 100 of horny layer protein are attached with identical reference signs, and detailed descriptions thereof are omitted.

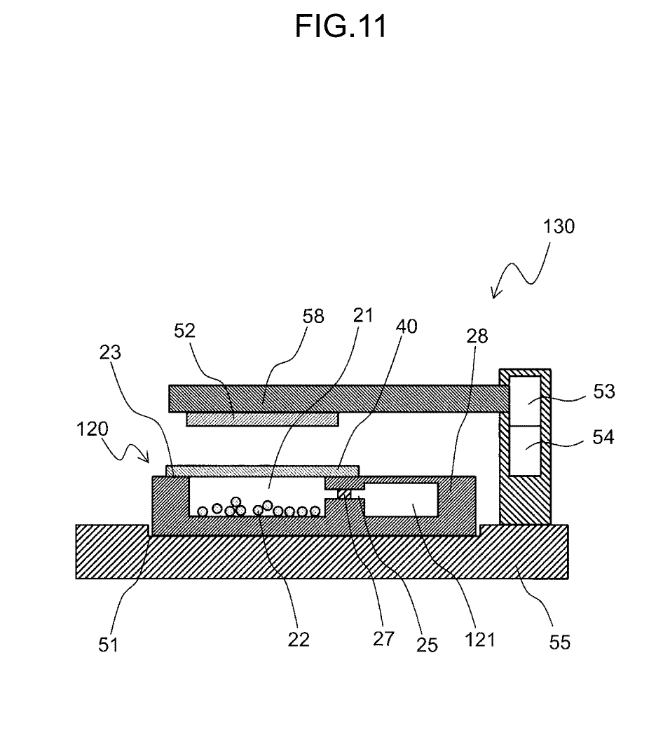

FIG. 11 is a schematic sectional view of the other example of the extraction system of horny layer protein according to the present exemplary embodiment.

Extraction container 120 used in extraction system 130 further includes analysis unit 121. Analysis unit 121 is connected to extraction unit 21 through recovery path 25. Recovery path 25 includes filter 27 for separating magnetic bodies 22 or impurities from the extraction solution. With this configuration, the extraction solution containing horny layer protein extracted in extraction unit 21 can easily be introduced into analysis unit 121.

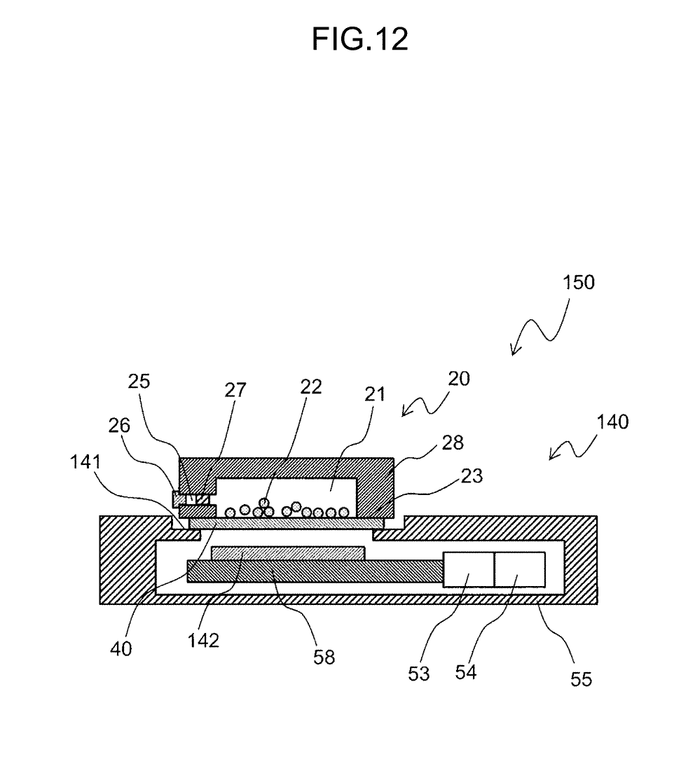

FIG. 12 is a schematic sectional view of still another example of the extraction system of horny layer protein according to the present exemplary embodiment.

In extraction system 150, extraction container 20 is disposed on extraction device 140, with sticking surface 23 being directed downward.

In extraction device 140, magnet 142 is provided on a lower side of placement part 141. A surface disposed with extraction container 20 has an opening. With the opening thus provided, magnet 142 can be positioned closer to collection tape 40.

With sticking surface 23 of extraction container 20 directed downward, collection tape 40 is positioned on a lower side of extraction unit 21. Therefore, the extraction reagent flowing into extraction unit 21 is reliably brought into contact with collection tape 40 and the skin tissue adhered to collection tape 40. Accordingly, even when extraction unit 21 is not completely filled with the extraction reagent, horny layer protein can effectively be extracted from the skin tissue.

The extraction method and the extraction system of horny layer protein according to one or more aspects have been described above based on the exemplary embodiment. However, the present disclosure is not limited to the exemplary embodiment. Configurations in which various variations conceived by those skilled in the art are applied to the present exemplary embodiment, and configurations established by combining structural elements in different exemplary embodiments may also fall within the scope of one or more aspects, without departing from the gist of the present disclosure.

### INDUSTRIAL APPLICABILITY

The present disclosure is particularly useful in extraction of horny layer protein that is performed as preprocessing for analysis or the like of protein contained in a horny layer of skin.

### REFERENCE MARKS IN THE DRAWINGS

20, 120: extraction container

21: extraction unit

22: magnetic substance

23: sticking surface

24: injection path

25: recovery path

26: stopper

27: filter

28: substrate

40: collection tape

41: horny layer collection part

42: adhesive part

50, 140: extraction device

51, 141: placement part

52, 142: magnet

53: drive unit

54: controller

55: plate

56: reagent tank

57: passage

58: support

100, 130, 150: extraction system

1. A biomolecule extraction method for extracting biomolecules contained in skin tissue using an extraction reagent and a magnetic substance in an extraction container, the biomolecule extraction method comprising:
bringing a tape adhered with the skin tissue into contact with the extraction reagent and the magnetic substance;
moving the magnetic substance in the extraction container by magnetic force generated from a magnet provided outside the extraction container; and
extracting the biomolecules from the skin tissue into the extraction reagent by mechanical friction between the magnetic substance and the tape adhered with the skin tissue.

2. The biomolecule extraction method according to claim 1, further comprising putting the extraction reagent into an extraction unit of the extraction container, after the tape is stuck on a sticking surface of the extraction container.

3. The biomolecule extraction method according to claim 2, further comprising:
bringing the skin tissue adhered to the tape into contact with the extraction reagent by disposing the extraction container such that the tape is under the extraction container after the tape is stuck on the sticking surface; and
bringing the magnetic substance into contact with the tape by moving the magnet below the extraction container.

4. The biomolecule extraction method according to claim 1, further comprising extracting the extraction reagent including the biomolecules from an extraction unit through a recovery path connected to the extraction unit of the extraction container.

5. The biomolecule extraction method according to claim 4, wherein the recovery path includes a filter, and separates the magnetic substance with the filter to recover the extraction reagent including the biomolecules.

6. A biomolecule extraction system comprising:
an extraction container configured to extract biomolecules from skin tissue;
an extraction device in which the extraction container is disposed; and
a carrier,
wherein the extraction container includes
an extraction unit configured to accommodate an extraction reagent of the biomolecules, and
a magnetic substance provided in the extraction unit,
the extraction device includes
a magnet configured to move the magnetic substance in the extraction unit, and
a controller configured to control movement of the magnet, and
the extraction device moves the magnetic substance in the extraction unit with the magnet, to extract the biomolecules from the skin tissue adhered to the carrier by mechanical friction generated between the magnetic substance and the carrier stuck with the skin tissue.

7. The biomolecule extraction system according to claim 6, wherein
the extraction container further includes a sticking surface to be stuck with the carrier that has collected the skin tissue,
the sticking surface includes an opening formed such that the skin tissue adhered to the carrier stuck on the extraction container faces the extraction unit,
the opening is formed so as to be entirely sealed by the carrier, and
the magnet in the extraction device is provided at a position facing the sticking surface.

8. The biomolecule extraction system according to claim 7, wherein the extraction device further includes an installation part configured to install the extraction container.

9. The biomolecule extraction system according to claim 6, wherein
the extraction device further includes
a support provided with the magnet, and
a drive unit configured to drive the support and the magnet, and
the controller controls an operation of the drive unit.

10. The biomolecule extraction system according to claim 6, wherein
the magnet in the extraction device is an electromagnet, and
the controller controls movement of the electromagnet with a current flowing through the electromagnet to move the magnetic substance through a change of a magnetic field generated by the electromagnet.

11. The biomolecule extraction system according to claim 8, wherein the magnet is under the installation part.

12. The biomolecule extraction system according to claim 6, wherein
the extraction device further includes a solution tank for accommodating the extraction reagent, and
the solution tank is connected to the extraction unit of the extraction container through a passage.

13. The biomolecule extraction system according to claim 6, wherein
the extraction container further includes
a recovery path connected to the extraction unit, and
a filter that is provided in the recovery path, and removes the magnetic substance from the extraction reagent.

14. The biomolecule extraction system according to claim 6, wherein the magnetic substance is fixed to an inside of the extraction unit with a water-soluble adhesive.

15. A biomolecule extraction container for extracting biomolecules from skin tissue adhered to the tape by mechanical friction force between a magnetic substance and a carrier, the biomolecule extraction container comprising:
a substrate;
a recess provided in the substrate; and
the magnetic substance provided in the recess.

16. The biomolecule extraction container according to claim 15, wherein the magnetic substance is fixed to an inside of the recess with a water-soluble adhesive.

17. The biomolecule extraction container according to claim 15, further comprising:
a recovery path connected to the extraction unit; and
a filter that is provided in the recovery path, and removes the magnetic substance from an extraction reagent.

18. A biomolecule extraction method for extracting biomolecules contained in skin tissue using an extraction reagent and a magnetic substance in an extraction container, the biomolecule extraction method comprising:
bringing a carrier adhered with the skin tissue into contact with the extraction reagent and the magnetic substance;
moving the magnetic substance in the extraction container by magnetic force generated from a magnet provided outside the extraction container; and
extracting the biomolecules from the skin tissue into the extraction reagent by mechanical friction between the magnetic substance and the carrier adhered with the skin tissue.

19. The biomolecule extraction method according to claim 18, wherein the carrier has a flat shape.

20. The biomolecule extraction method according to claim 18, wherein the carrier is a tape.
